(19) United States
(12) Patent Application Publication
(10) Pub. No.: US 2025/0035552 A1
(43) Pub. Date: Jan. 30, 2025

(54) GEL PARTICLES WITH TAGS FOR LABELING DRILL CUTTINGS

(71) Applicant: Saudi Arabian Oil Company, Dhahran (SA)

(72) Inventors: Raed Z Makeen, Cambridge, MA (US); S. Sherry Zhu, Cambridge, MA (US); Saleh Altuwayjiri, Cambridge, MA (US); Sehoon Chang, Cambridge, MA (US); Marta Antoniv, Cambridge, MA (US); Gawain Thomas, Cambridge, MA (US)

(21) Appl. No.: 18/361,387

(22) Filed: Jul. 28, 2023

(51) Int. Cl. G01N 21/64; G01N 33/28

(52) U.S. Cl. CPC G01N 21/6428; G01N 33/2823; G01N 2021/6441

(57) ABSTRACT

The disclosure relates to compositions, methods and systems that include gel particles that include a tag. The gel particles can be used to label drill cuttings generated during a drilling operation.

### FIELD

The disclosure relates to compositions, methods and systems that include gel particles that include a tag. The gel particles can be used to label drill cuttings generated during a drilling operation.

### BACKGROUND

Tags can be used to label downhole drill cuttings generated during drilling operations. Such tags can be introduced into the formation using a transporting fluid (e.g., a drilling fluid, a fracturing fluid, a completion fluid).

### SUMMARY

The disclosure relates to compositions, methods and systems that include gel particles that include a tag. The gel particles can be used to label drill cuttings generated during a drilling operation.

The compositions, methods and systems can provide a relatively accurate analysis of petrophysical and/or petrochemical properties of a formation, relative to certain other compositions, methods and systems.

The methods may involve fewer processing steps (e.g., washing, extraction, column separation) to isolate the tags from cuttings relative to certain other compositions, methods and systems for labeling cuttings. Additionally, the sample analysis may be less expensive, more robust, easier to operate and implement in-field and safer relative to certain other methods for tagging cuttings.

The gel particles can form upon contact with a drilling fluid (e.g., oil-based mud, water-based mud) without the use of a manufacturing facility. In some embodiments, the tags can be incorporated into the gel particles via hydrogen bonding, which can be conducted in situ and is simpler relative to certain other methods, such as methods that include covalently attaching the tag to the polymers.

In a first aspect, the disclosure provides a method, including: providing a first liquid including a polymer and a first tag; providing a drilling fluid including an oil-based mud and/or a water-based mud; combining the first liquid with the drilling fluid to form a second liquid including gel particles disposed in the drilling fluid, the gel particles including the polymer and the first tag; and flowing the second liquid into an underground formation during drilling of the underground formation at a first time.

In some embodiments, the gel particles attach to cuttings formed while drilling the underground formation.

In some embodiments, the method further includes: collecting the cuttings; providing an excitation source to the cuttings; and based on whether a fluorescence signal is measured from the cuttings, determining whether gel particles including the first tag are present on the cuttings.

In some embodiments, the method further includes: collecting the cuttings; extracting the first tag from the gel particles attached to the cuttings to form a third liquid including the first tag; measuring a fluorescence of the third liquid; and based on the measured fluorescence of the third liquid, determining a depth at which the cuttings were formed.

In some embodiments, the drilling fluid includes an oil-based mud and the gel particles have a size of 100 μm to 2 mm.

In some embodiments, the drilling fluid includes a water-based mud and the gel particles have a size of 1 mm to 5 mm.

In some embodiments, the gel particles include the first tags hydrogen-bonded to the polymer.

In some embodiments, a concentration of the first tags in the gel particles is 20 ppm to 100 ppm.

In some embodiments, a ratio of the drilling fluid to the first liquid is from 10:1 to 1000:1.

In some embodiments, the polymer includes hydroxyl groups.

In some embodiments, the polymer has a molecular weight of 100 kD to 500 kD.

In some embodiments, the polymer includes a polyvinyl alcohol (PVOH), a modified PVOH, a PVOH copolymer, a modified PVOH copolymer, a guar gum, a polysaccharide, a polyacrylamide, a polyacrylic acid, and a butendiol vinyl alcohol (BVOH).

In some embodiments, the first tag includes a dye, a metal complex and/or a halide-substituted organic acid.

In some embodiments, the method further includes: providing a fourth liquid including a polymer and a second tag; providing the drilling fluid; combining the fourth liquid with the drilling fluid to form a fifth liquid including gel particles disposed in the drilling fluid, the gel particles including the polymer and the second tag; and flowing the fifth liquid into an underground formation during drilling of the underground formation at a second time. The second tag is different from the first tag and the second time is different from the first time.

In some embodiments, the gel particles include the second tag attach to cuttings formed while drilling the underground formation.

In some embodiments, the method further includes: collecting the cuttings; providing an excitation source to the cuttings; and based on whether a fluorescence signal is measured from the cuttings, determining whether gel particles including the second tag are present on the cuttings.

In some embodiments, the method further includes: collecting the cuttings; extracting the second tag from the gel particles attached to the cuttings to form a sixth liquid including the second tag; measuring a fluorescence of the sixth liquid; and based on the measured fluorescence of the sixth liquid, determining a depth at which the cuttings were formed.

In some embodiments, the second liquid is flowed into the underground formation via a drill pipe.

In a second aspect, the disclosure provides a system, including: a first liquid including a polymer; a second liquid including a tag; a mixer configured to mix the first and second liquids to provide a third liquid including the polymer and the tag; and a drill configured to drill a borehole into an underground formation. The drill includes a line configured to circulate a drilling fluid in the borehole, and the drilling fluid includes a water-based mud and/or an oil-based mud. An output of the mixer is in fluid communication with the line so that, during use of the system: the third liquid flows from the mixer to the line to combine the third liquid and the drilling fluid; combining the third liquid and the drilling fluid forms a fourth fluid which includes gel particles; and the gel particles include the polymer bonded with the tag.

In certain embodiments, the drill forms cuttings during the drilling and the drilling fluid circulates the cuttings to a surface above the underground formation and the gel particles attach to at least a portion of the cuttings.

### BRIEF DESCRIPTION OF THE FIGURES

FIG. 1 depicts a schematic of a method.

FIG. 2 schematically depicts a portion of a gel particle with hydrogen bonding between hydroxyl groups of a polymer and tags.

FIG. 3 depicts a schematic of a system.

FIG. 4A shows a photograph of gel particles with tags in oil-based mud under excitation of an ultraviolet (UV) lamp.

FIG. 4B shows a photograph of fluorescent gel particles with tags on limestone particles visualized with a UV lamp.

FIG. 4C shows a photograph of fluorescent gel particles with tags on limestone particles visualized with a fluorescence microscope.

FIG. 4D shows a photograph of fluorescent gel particles in an oil-based mud visualized with a fluorescence microscope

FIG. 5 depicts a bar graph of the emission intensity of different samples.

FIG. 6 depicts a schematic of a method.

### DETAILED DESCRIPTION

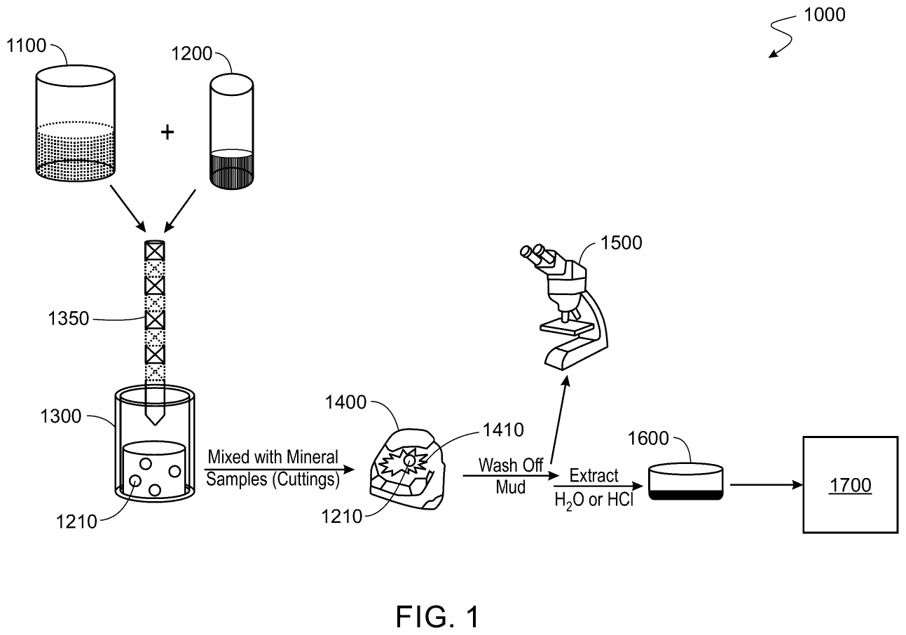

FIG. 1 depicts a schematic of a method 1000. In the method 1000, a first liquid 1100 containing a polymer is mixed with a second liquid 1200 containing tags 1210 to form a liquid 1300 containing the polymer and the tags. In some embodiments, the liquids 1100 and 1200 are mixed using a mixing device (e.g., a static mixer, a vortex mixer, an overhead mixer) 1350 to form the liquid 1300. In certain embodiments, the tags 1210 are distributed relatively uniformly throughout the liquid 1300.

Without wishing to be bound by theory, it is believed that when the liquid 1300 is added to a drilling fluid, such as an oil-based mud or water-based mud, gel particles 1410 form in-situ in the drilling fluid due to the immiscibility of the liquid 1300 and the drilling fluid. Also without wishing to be bound by theory, it is believed that tags 1210 are confined within the gel particles 1410 due to hydrogen bonding and/or polar interactions between the tags 1210 and the polymer, such as through hydroxyl groups of the polymer. Without wishing to be bound by theory, it is believed that polar interactions can confine the tags inside gel particle if the polar interactions are stronger than any interactions between the tag and the fluid outside the gel particles. Gel particles with confined tags can form in aprotic organic solvents such as hexane, oil-based muds and water-based muds with a relatively high salinity.

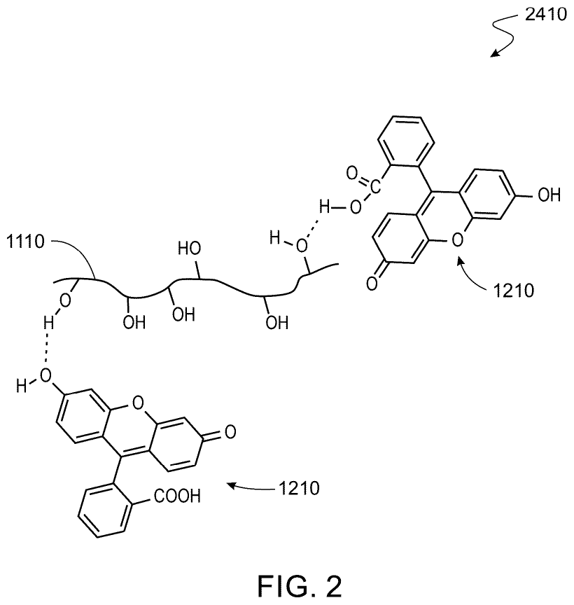

FIG. 2 schematically depicts a portion of a gel particle 2410 in which the hydroxyl groups of a polymer 1110 are hydrogen bonded with a tag 1210 (fluorescein). Without wishing to be bound by theory, it is believed that the hydrogen bonding strength of the polymer 1110 allows it to form gel particles 1410 upon contact with the drilling fluid.

Without wishing to be bound by theory, it is believed that immiscibility between the polymer solution and the mud can cause the phase separation of the aqueous polymer solution from the mud. The shape of the phase-separated-gel polymer solution formed in mud generally depends on the interfacial tension between the polymer solution and the mud, where the final shape is a result of the lowest free energy.

In general, the polymer 1110 is selected such that it can form gel particles with the tag 1210 incorporated within the gel particle when contacted with a mud (e.g., an oil based mud, a water based mud). Polymers that are not soluble in water (e.g., modified starch or dextran) may be immiscible with the tag 1210. Additionally, certain polymers (e.g., dextran) do not form gel particles when contacted with a mud (e.g., an oil-based mud).

Referring again to FIG. 1, when the drilling fluid containing the gel particles 1410 is contacted with cuttings 1400 resulting from a drilling operation (see discussion below), the gel particles 1410 can adhere to the cuttings 1400. Fluorescence microscopy 1500, or any other method suitable for detecting the presence of the tags 1210, is used to determine whether a cutting 1400 includes the gel particles 1410. Cuttings 1400 identified as bearing the gel particles 1410 can be washed to remove mud, resulting in cuttings 1400 bearing the gel particles 1410 that are free from mud. The tags can be extracted from the washed cuttings 1400 using an appropriate agent (e.g., water or acid, such as HCl) to provide a liquid 1600 that includes the tags 1210. The liquid 1600 can be analyzed using a fluorescence-measuring instrument, such as a fluorescence microplate reader 1700 to determine the identity of the tags 1210. The identity of the tags 1210 can be used to determine a depth at which the cuttings 1400 were generated by the drill bit (see discussion below).

In general, the polymer is water-soluble and includes hydrophilic groups such as hydroxyl, carboxylic, and/or amide groups that can form hydrogen bonds. Examples of the polymer include polyvinyl alcohols (PVOH), modified PVOH, a PVOH copolymer, a modified PVOH copolymer (e.g., Nichigo G-polymer), guar gums, polysaccharides, polyacrylamides, polyacrylic acids, Nichigo G-polymer, and butendiol vinyl alcohols (BVOH). Without wishing to be bound by theory, it is believed that the polymer 1110 can form viscous solutions in aqueous fluids and form gel particles in drilling fluids. In some embodiments, the polymer is at least 4 (e.g., at least 5, at least 10, at least 15) wt. % and/or at most 20 (e.g., at most 15, at most 10) wt. % of the first liquid 1100. In some embodiments, the molecular weight of the polymer is 100 (e.g., at least 200, at least 300, at least 400) (kiloDaltons) kD and/or at most 500 (e.g., at most 400, at most 300, at most 200) kD.

In some embodiments, the polymer is Nichigo G-polymer. G-polymer (supplied by Soarus LLC, Mitsubishi Chemical) is a relatively low crystallinity, relatively low melting point, extrudable, and biodegradable polymer with relatively high hydrogen bonding strength. G-polymer is a modified polyvinyl alcohol copolymer that contains hydroxyl groups. G-polymer is soluble in water and heavy brines at room temperature, forming a viscous polymer solution with little shear-thinning. Solutions of G-polymer also have a relatively high surface tension and are relatively stable in water (they do not precipitate), relative to certain other polymer solutions.

In general, the tags 1210 are detectable by any of a variety of techniques, such as fluorescence, X-ray fluorescence (XRF), GCMS, UV-VIS, and/or FTIR. In general, the tags 1210 are soluble in water or dispersible in the aqueous polymer solution (the first liquid 1100). Examples of the tags 1210 include dyes, metal complexes, polymeric nanoparticles, and halide-substituted organic acids. Example of water-soluble dyes suitable for use as tags include rhodamine dyes, fluorescein, Nile blue, yellow-CF405, CF640R, CF405S, CF350, and pigments based on rare-earth metal ions such as lanthanide and europium ions. Examples of halide-substituted organic acids include chlorobenzoic acid and bromobenzoic acid. Examples of polymeric nanoparticles including polychlorostyrene and polybromostyrene.

In certain embodiments, a tag 1210 is a metal complex. In general, metal complexes are selected that can be detected by X-ray fluorescence (XRF) including an aluminum oxide, a titanium oxide, an iron oxide, a zinc oxide, a tungsten oxide, a ruthenium oxide, MoO₃, CuO, SnO₂, ZrO₂, CoO, CrO₃, and Mn₂O₇. Metal complexes can be disposed in an aqueous solution, as shown in FIG. 1, or can be added directly to the first liquid 1100 prior to addition to the drilling fluid.

In certain embodiments, the concentration of the tags 1210 in the second liquid 1200 is at least 1 (e.g., at least 2, at least 3, at least 4, at least 5, at least 6, at least 7, at least 8, at least 9, at least 10, at least 20, at least 30, at least 40, at least 50, at least 60, at least 70, at least 80, at least 90) ppm and/or at most 100 (e.g., at most 90, at most 80, at most 70, at most 60, at most 50, at most 40, at most 30, at most 20, at most 10, at most 9, at most 8, at most 7, at most 6, at most 5, at most 4, at most 3, at most 2) ppm.

Without wishing to be bound by theory, it is believed that the size of the gel particles 1410 is determined by the type of mud (drilling fluid). In oil-based mud, the gel particles 1410 are typically on the order of several hundred microns in size. In water-based mud, the gel particles 1410 are usually on the order of millimeters in size. In certain embodiments, the size of the gel particles 1410 formed in an oil-based mud are at least 100 (e.g., at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900) μm and/or at most 2000 (e.g., at most 1900, at most 1800, at most 1700, at most 1600, at most 1500, at most 1400, at most 1300, at most 1200, at most 1100, at most 1000, at most 900, at most 800, at most 700, at most 600, at most 500, at most 400, at most 300, at most 200) μm. In certain embodiments, the size of the gel particles 1410 formed in a water-based mud are at least 1 (e.g., at least 1.5, at least 2, at least 2.5, at least 3, at least 3.5, at least 4, at least 4.5) mm and/or at most 5 (e.g., at most 4.5, at most 4, at most 3.5, at most 3, at most 2.5, at most 2, at most 1.5) mm. In general, an oil-based mud can include diesel, water, surfactants clay and salts. In general, a water-based mud can include water, clay, salts and surfactants.

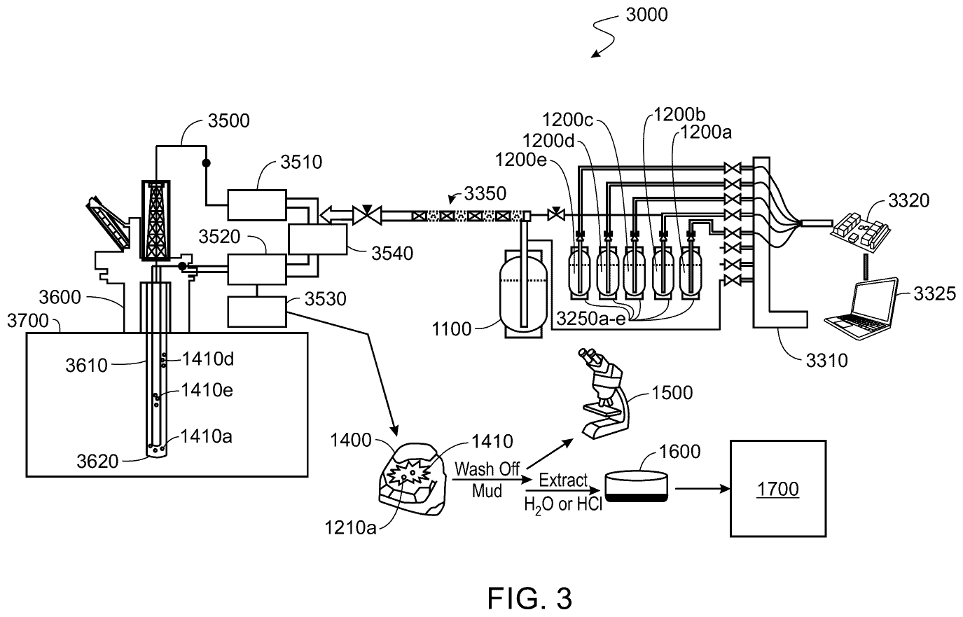

FIG. 3 depicts a schematic for a system 3000. In the system 3000, the liquid 1100 is made by disposing the polymer in an aqueous solution. First, second, third, fourth and fifth tags 1210a, 1210b, 1210c, 1210d, and 1210e, each with a different detection property, such as a different fluorescence property (e.g., emission wavelength), XRF property, or GCMS signal, are disposed in an aqueous solution to form corresponding tag-containing liquids 1200a-1200e. Tag-containing liquids 1200a-1200e are disposed in corresponding separate tanks 3250a-3250e. A computer 3320 with control software 3325 controls injection of the tag-containing liquids 1200a-1200e and the liquid 1100 into an inline mixer (e.g., a static mixer) 3350 at a fixed ratio. Typically, the ratio is determined based on a desired final concentration of the dye in the drilling fluid and the drilling fluid flow rates. An air supply 3310 is used to pressurize the tanks 3250a-e to inject the tag-containing liquids 1200a-1200e. In certain embodiments, the ratio of first liquid 1100 to a given the tag-containing liquids 1200a-1200e is at least 10:1 (e.g., at least 20:1, at least 50:1, at least 100:1, at least 200:1, at least 500:1) and/or at most 1000:1 (e.g., at most 500:1, at most 200:1, at most 100:1, at most 50:1, at most 20:1). The mixer 3350 mixes (e.g., thoroughly mixes) the liquid 1100 and the given tag-containing liquid 1200a-1200e before entering the line 3500. The line 3500 includes drilling fluid (e.g., oil-based mud, water-based mud). When the liquid 1100 and the given tag-containing liquid 1200a-1200e contact the drilling fluid in the line 3500, corresponding gel particles 1410a-1410e are formed. In certain embodiments, the concentration of the given gel particles 1410 in the drilling fluid is 0.1 (e.g. at least 0.2, at least 0.5, at least 1, at least 2, at least 5) % and/or at most 10 (e.g., at most 5, at most 2 at most 1, at most 0.5, at most 0.2).

A high-pressure (“HP”) system 3510 increases the pressure of the drilling fluid prior to injection into a drill 3600. The drill 3600 includes a drill pipe 3610 that extends into an underground formation 3700 and a drill bit 3620 to drill into the underground formation 3700. As the drill bit 3620 cuts into the underground formation 3700, cuttings 1400 are generated.

The gel particles 1410 in the drilling fluid are carried to the drill bit 3620. The gel particles 1410 can adhere to a cutting 1400, thereby tagging the cutting 1400 with the corresponding tag 1210a-1210e. The different tags 1210a-1210e can be introduced at different times during the drilling process (corresponding to different depths of drilling) to form gel particles 1410 with different tags 1210a-1210e to tag the cuttings 1400 based on depth. For example, by introducing the tags 1210a, 1210d and 1210e at first, second and third times respectively, gel particles 1410a, 1410d and 1410e can be formed which will tag the cuttings 1400 formed at first, second and third depths, respectively. Without wishing to be bound by theory, it is believed that, in some embodiments, the depths can be determined using the time of injecting the tags 1200a-1200e, the lag time for the tags 1200a-1200e to travel down the drill pipe 3610 to the drill bit 3620 (calculated using mud volume, mud flow rate and volume of the drill pipe 3610) and the depth of the drill bit 3620 at the injection time plus lag time.

In general, the concentration of tags 1210a-1210e in the drilling fluid is less than 10 ppm; however, the concentration of tags in the gel particles 1410 is typically 20-100 ppm. In some embodiments, the concentration of tags in the gel particles 1410 is at least 20 (e.g., at least 30, at least 40, at least 50, at least 60, at least 70, at least 80, at least 90) ppm and/or at most 100 (e.g., at most 90, at most 80, at most 70, at most 60, at most 50, at most 40, at most 30) ppm.

The cuttings 1400 are subsequently carried upward and out of the drill pipe 3610 by the drilling fluid, after which the cuttings 1400 are collected. In some embodiments, this involves passing the drilling fluid through a mud treatment system 3520 and an active tank 3540 before the drilling fluid is recirculated back to the drill 3600. The active tank 3540 holds the drilling fluid that is circulating. The cuttings 1400 are passed to a shale shaker 3530.

The cuttings 1400 are collected from the cutting handling system (shale shaker 3530) and processed as described in FIG. 1. The mud is washed away first and then fluorescence microscopy 1500, or any other method suitable for detecting the presence of the tags 1210, can be used to determine whether a cutting 1400 includes a given gel particle 1410a-1410e. The given tag 1210a-1210e can be extracted using water or acid (e.g., HCl) to provide the liquid 1600 that includes the given tag 1210a-1210e. The liquid 1600 can be analyzed (e.g., in a fluorescence microplate reader 1700) to determine the identity of the given tag 1210a-1210e. The identity of the tags 1210a-1210e can be used to determine a depth at which the cuttings 1400 were generated based on when the given tag 1210a-1210e was introduced.

### EXAMPLE

### Example 1

3.5 g of G-polymer 1009 (supplied by Soarus LLC, Mitsubishi Chemical) was slowly added to 50 ml of water or a brine to make a 7% w/v solution. The mixture was stirred overnight at room temperature to dissolve the polymer. 2.5 mg of Rhodamine B powder or fluorescein powder (tag) was added to 50 mL of water under stirring to make a 50 ppm dye stock solution. The dye solution was diluted to 20, 10 and 5 ppm using the G1009 polymer solution as shown in Table 1.

TABLE 1
Dilution table for tag in polymer solution.

| Final | | | Volume |
|---|---|---|---|
| fluorescein tag | Volume of | Volume of | of final |
| concentration | fluorescein | additional | fluorescein |
| in polymer | stock | G1009 | polymer |
| solution | solution | solution | solution |
| (ppm) | (50 ppm) | (mL) | (mL) |
| 20 | 4 | 6 | 10 |
| 10 | 2 | 8 | 10 |
| 5 | 1 | 9 | 10 |

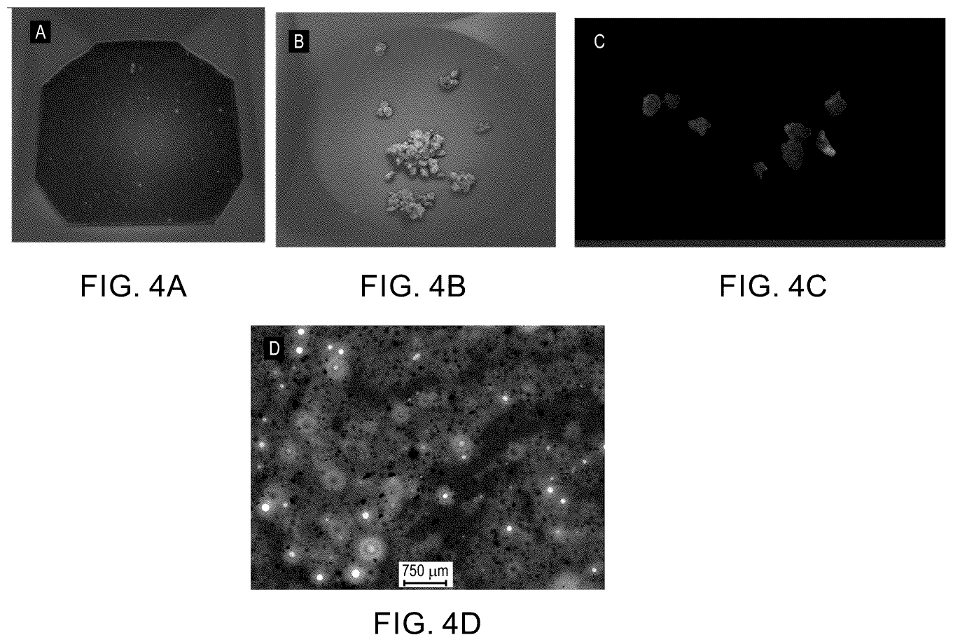

0.2 mL of polymer gel solution containing 5, 10, or 200 ppm fluorescein dye was added dropwise to 2 mL of oil-based mud obtained from the field under stirring to form gel particles with tags inside the gel particles for a final dye concentration of 0.45, 1.1, and 1.8 ppm in mud, respectively. The dye was confined inside the gel particles (gel-tag) which were dispersed in the mud. FIG. 4A shows the gel particles with fluorescent tags (green dots), under excitation from a UV lamp (365 nm) in the mud, formed using the 10 ppm tag in polymer solution.

0.5 g of limestone particles were added to each mud sample and the samples were shaken at 200 rpm for over 12 hours. 2 mL of hexane was added to the vial containing the mud/limestone samples, and the top mixture of mud and hexane were carefully decanted to leave the limestone particles at the bottom of the vial (hexane washing). The hexane-washing step was repeated two more times or until the limestone particles appeared mud-free. Hexane was evaporated under vacuum to give dry limestone particles with gel tags. FIGS. 4B and 4C show the gel particles with fluorescein visible on dried limestone particles, visualized under a UV lamp and fluorescence microscope, respectively.

### Example 2

G1009 polymer was dissolved in two brines (LS brine and SW brine) with different salinities to make 7% polymer solution as described in Example 1. Both brines contained NaCl, CaCl₂), MgCl₂ and NaHCO₃, and the LS brine had a higher salinity than the SW brine. 5, 10, and 20 ppm of rhodamine in the two polymer solutions were prepared as described in Example 1. 0.2 mL of each rhodamine in polymer solution was added to 2 mL of oil-based mud to form gel particles in mud that contained 0.45-1.8 ppm of rhodamine dye. FIG. 4D shows a photograph of fluorescent gel particles in an oil-based mud visualized with a fluorescence microscope The dye was confined inside the gel particles with tags that were dispersed in the mud (FIG. 4D). No fluorescence was observed in the oil-based mud without the gel particles with tags when viewed under the fluorescence microscope. The experimental protocol of Example 1 was used to label the limestone particles using the rhodamine and polymer solution. After recovering the limestone particles labeled with gel particles with rhodamine, the dye was extracted from the limestone particles using either water or by dissolving the dye and the limestone in 15% HCl. Fluorescence of the dye solution was analyzed using a microplate reader.

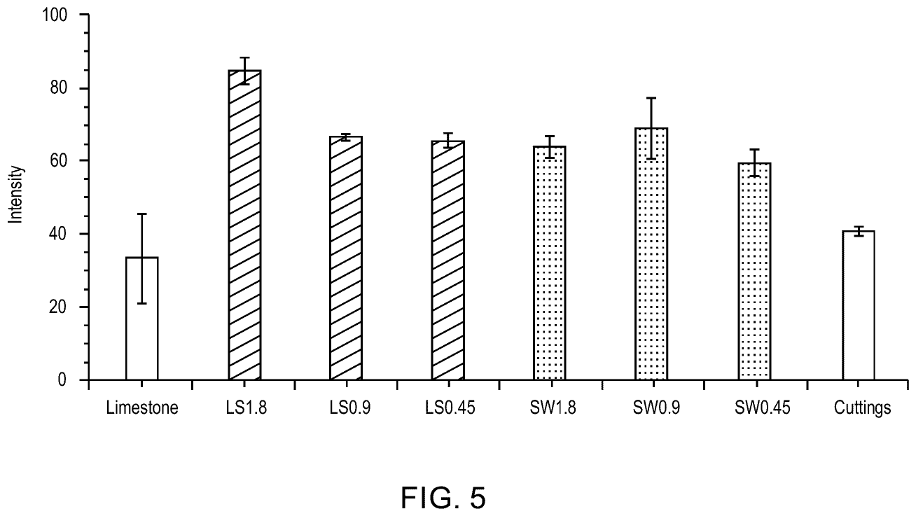

FIG. 5 shows the fluorescence intensity of gel particles with rhodamine (5, 10 or 20 ppm dye inside gel particles) on limestone particles recovered from oil-based mud containing a final concentration of 0.45, 0.9 or 1.8 ppm dye in the LS and SW brines. The samples were excited at 550 nm and the fluorescence intensities were recorded at 585 nm using a microplate reader. Each microplate reader measurement for fluorescence was repeated three times for each sample. Three aliquots of liquid were loaded into three different wells in a 96-well plate, and an average of the three measurements was calculated. Compared to the limestone control and the cutting samples, which were recovered from oil-based mud without any gel particles, all tagged limestone samples displayed a higher emission intensity at 585 nm, indicating the presence of dye originating from gel particles with rhodamine.

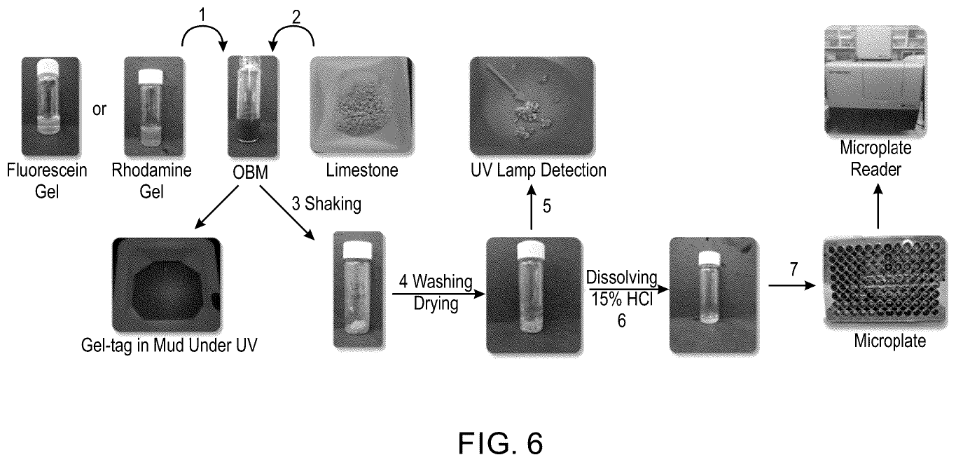

The procedures employed in Examples 1 and 2 are summarized in FIG. 6. In step 1, solution with the polymer and dye (fluorescein or rhodamine) was added to the oil-based mud (OBM). In step 2, the limestone particles were added to the oil-based mud. In step 3, the sample was shaken overnight. In step 4, the mud was washed off using hexane and the limestone samples were dried. In step 5, the gel particles with tags were detected under a UV lamp. In step 6, the gel particles with tags on limestone particles were dissolved using water or 15% HCl. In step 7, the gel particles with tags were detected using a microplate reader.

1. A method, comprising:
providing a first liquid comprising a polymer and a first tag;
providing a drilling fluid comprising a member selected from the group consisting of an oil-based mud and a water-based mud;
combining the first liquid with the drilling fluid to form a second liquid comprising gel particles disposed in the drilling fluid, the gel particles comprising the polymer and the first tag; and
flowing the second liquid into an underground formation during drilling of the underground formation at a first time.

2. The method of claim 1, wherein the gel particles attach to cuttings formed while drilling the underground formation.

3. The method of claim 2, further comprising:
collecting the cuttings;
providing an excitation source to the cuttings; and
based on whether a fluorescence signal is measured from the cuttings, determining whether gel particles comprising the first tag are present on the cuttings.

4. The method of claim 2, further comprising:
collecting the cuttings;
extracting the first tag from the gel particles attached to the cuttings to form a third liquid comprising the first tag;
measuring a fluorescence of the third liquid; and
based on the measured fluorescence of the third liquid, determining a depth at which the cuttings were formed.

5. The method of claim 1, wherein:
the drilling fluid comprises an oil-based mud; and
the gel particles have a size of 100 μm to 2 mm.

6. The method of claim 1, wherein:
the drilling fluid comprises a water-based mud; and
the gel particles have a size of 1 mm to 5 mm.

7. The method of claim 1, wherein the gel particles comprise the first tags hydrogen-bonded to the polymer.

8. The method of claim 1, wherein a concentration of the first tags in the gel particles is 20 ppm to 100 ppm.

9. The method of claim 1, wherein a ratio of the drilling fluid to the first liquid is from 10:1 to 1000:1.

10. The method of claim 1, wherein the polymer comprises hydroxyl groups.

11. The method of claim 1, wherein the polymer has a molecular weight of 100 kD to 500 kD.

12. The method of claim 1, wherein the polymer comprises a member selected from the group consisting of a polyvinyl alcohol (PVOH), a modified PVOH, a PVOH copolymer, a modified PVOH copolymer, a guar gum, a polysaccharide, a polyacrylamide, a polyacrylic acid, and a butendiol vinyl alcohol (BVOH).

13. The method of claim 1, wherein the first tag comprises a member selected from the group consisting of a dye, a metal complex and a halide-substituted organic acid.

14. The method of claim 1, further comprising:
providing a fourth liquid comprising a polymer and a second tag;
providing the drilling fluid;
combining the fourth liquid with the drilling fluid to form a fifth liquid comprising gel particles disposed in the drilling fluid, the gel particles comprising the polymer and the second tag; and
flowing the fifth liquid into an underground formation during drilling of the underground formation at a second time,
wherein:
the second tag is different from the first tag; and
the second time is different from the first time.

15. The method of claim 14, wherein the gel particles comprising the second tag attach to cuttings formed while drilling the underground formation.

16. The method of claim 15, further comprising:
collecting the cuttings;
providing an excitation source to the cuttings; and
based on whether a fluorescence signal is measured from the cuttings, determining whether gel particles comprising the second tag are present on the cuttings.

17. The method of claim 15, further comprising:
collecting the cuttings;
extracting the second tag from the gel particles attached to the cuttings to form a sixth liquid comprising the second tag;
measuring a fluorescence of the sixth liquid; and
based on the measured fluorescence of the sixth liquid, determining a depth at which the cuttings were formed.

18. The method of claim 1, wherein the second liquid is flowed into the underground formation via a drill pipe.

19. A system comprising:
a first liquid comprising a polymer;
a second liquid comprising a tag;
a mixer configured to mix the first and second liquids to provide a third liquid comprising the polymer and the tag; and
a drill configured to drill a borehole into an underground formation, the drill comprising a line configured to circulate a drilling fluid in the borehole, the drilling fluid comprising a member selected from the group consisting of a water-based mud and an oil-based mud,
wherein:
an output of the mixer is in fluid communication with the line so that, during use of the system:
the third liquid flows from the mixer to the line to combine the third liquid and the drilling fluid;
combining the third liquid and the drilling fluid forms a fourth fluid which comprises gel particles; and
the gel particles comprises the polymer bonded with the tag.

20. The system of claim 19, wherein:
the drill forms cuttings during the drilling and the drilling fluid circulates the cuttings to a surface above the underground formation; and
the gel particles attach to at least a portion of the cuttings.
